(19) United States
(12) Patent Application Publication
(10) Pub. No.: US 2019/0247360 A1
(43) Pub. Date: Aug. 15, 2019

(54) Pharmaceutical Composition for Treating Bacterial and Viral infections

(71) Applicant: Edward Wick, Sussex, WI (US)

(72) Inventor: Edward Wick, Sussex, WI (US)

(21) Appl. No.: 16/153,589

(22) Filed: Oct. 5, 2018

Related U.S. Application Data
(63) Continuation-in-part of application No. 15/987,649, filed on May 23, 2018, now Pat. No. 10,166,265.
Application No. 15/987,649 is a continuation-in-part of application No. 15/893,199, filed on Feb. 9, 2018, now Pat. No. 10,149,831.

(51) Int. Cl. A61K 31/375; A61K 31/07; A61K 31/593; A61K 31/4415; A61K 33/30; A61K 36/8962; A61P 31/04; A61P 31/16

(52) U.S. Cl. CPC A61K 31/375; A61K 31/07; A61K 31/593; A61K 31/4415; A61K 9/20; A61K 36/8962; A61P 31/04; A61P 31/16; A61K 33/30

(57) ABSTRACT

The present invention relates to a pharmaceutical; composition comprising an effective amount of vitamin C, zinc, vitamin A, vitamin D3, vitamin B6, and garlic to treat bacterial and/or viral infections, and a method for treating bacterial and/or viral infections by orally administering the composition to a patient effective to reduce symptoms of bacterial and/or viral infections such a cold and influenza.

### FIELD OF INVENTION

The present invention relates generally to a pharmaceutical composition and its use. More specifically, the present invention is a pharmaceutical composition comprising an effective amount of vitamin C, vitamin, zinc, vitamin A, vitamin D3, B6, and garlic, to treat bacterial or viral infection, and a method for treating bacterial or viral infection by orally administrating the composition to a patient effective to reduce the symptoms of bacterial and/or viral infections such as cold or influenza.

### BACKGROUND OF INVENTION

To treat bacterial infections a variety of antibiotics drugs are available. However, the over-prescription of antibiotics over the past half century give rise to antibiotic resistant strains of bacteria such as superbugs (1). More than 400 kinds of superbugs are known to cause human diseases. These diseases include, for example the common cold, influenza, cold soars (Herpes type 1), genital herpes (Type 2), norovirus, mononucleosis, shingles, hepatitis, dengue, West Nile fever, severe acute respiratory syndrome (SARS), Hantavirus, Ebola, and acquired immunodeficiency syndrome (AIDS), especially in cases of the common cold and influenza, rapid mutations make it difficult for the body immune system to identify and react to the invading viruses quickly (1). For viral infections in general, and upper respiratory viral infections in particular, effective drugs to hinder reproduction of the infectious agents are much less available. Prescription of anti-flu drugs currently available may reduce complications such as pneumonia, and often viruses are resistant to drugs. As an alternative to conventional pharmaceuticals for treating bacterial or viral infection, interest has grown in the use of certain food supplements for the enhancing or stimulating the immune system in the human body (2). One object of the present invention is to use and alternative composition to enhance or stimulate the immune system of the human body, thus to aid the body's ability to self-repair.

Wellness can be achieved with the behavioral and lifestyle modifications utilizing multimodal approaches. For example, with proper diet, especially vegetables, exercise, and botanical alternatives, this approach strives to improve the physical and emotional wellness of patients. The present inventor has identified certain alternatives. The inclusion of prevention and wellness management by physicians as a standard of patient care will decrease in medical errors and will contribute significantly in lowering the cost of health care.

It is therefore an aim of the present invention to provide and alternative composition for treating bacterial or viral infection in a patient, the alternative composition comprising an effective amount of vitamin C, zinc, vitamin A, vitamin D3, Vitamin B6, and garlic; a method for treating bacterial or viral infection in a patient effective to reduce the symptoms of bacterial and/or viral infections such as colds and influenza.

### DETAIL DESCRIPTION OF THE INVENTION

The present invention is a oral composition comprising an effective amount of vitamin C, zinc, vitamin A, vitamin D3, vitamin B6, and garlic, that is able to treat bacterial or viral infection in a patient. The oral composition is utilized by either adults or children. The oral composition incorporates active ingredients which function cooperatively to enhance or stimulate the immune system of the human body in order to treat bacterial or viral infection.

Vitamin C (ascorbic acid), is a water-soluble vitamin found in fruit and vegetables, such as citrus fruit (3). It is necessary for the iron absorption, wound healing and collagen formation. Vitamin C is also recognized as being important for the successful productions of neurotransmitters and improvement of glucose metabolism, its deficiency results in neurological disease of scurvy. Vitamin C, which is associated with immune strengthening, is derived from its ability to enhance the function of the immune system, including antimicrobial and NK cell activities, macrophages, lymphocyte proliferation, chemotaxis, and delayed-type hypersensitivity (3). The recommended daily intake by the US food and Nutrition Board of the Institute of Medicine for men more than 18 years old is 90 mg of vitamin C daily; for women more than 18 years old, it is 75 mg daily (4). For treating the common cold, 200 mg to 3 g have been taken by mouth daily for three to five days or longer (4).

Zinc salts have been found to inhibit rhinovirus replication in vitro at concentrations of <0.1 mmole/L, possibly by interfering with rhinovirus protein cleavage (3). It also has been suggested that zinc salts may protect plasma membranes against lysis by cytotoxic agents such as microbial toxins and complement. The proposed protective mechanism is either via immunomodulation or via the binding of zinc ions to rhinovirus surface canyons, thus inhibiting viral interactions with intercellular adhesions molecule-1 (ICAM-1), the site of rhinovirus binding to cells. Because ICAM-1 is also the binding site for leukocyte function associated antigen-1 (LEA-1), the block of LFA-1/ICAM-1 binding has been postulated to possibly suppress inflammation. Several randomized, controlled clinical studies showed a beneficial effect of using zinc for treating the common cold, particularly when zinc is started within the first 24 hours of onset of symptoms (3). The current recommended dietary allowance for zinc taken by mouth is: 11 mg for males 19 years and older; and 8 mg for females 19 years and older (5). For the common cold, doses have ranged from 4.5-24 mg of zinc (gluconate or acetate) in the form of lozenges taken by mouth every 1-3 hours for 3-14 days or until symptoms resolved.

Vitamin A, use as a dietary supplement, for adults and teenagers: oral dosage form (capsules or chewable tablets) has 6-15 mg of beta-carotene (the equivalent of 10.000-25000 units of vitamin A activity) per day (6). For children, the oral dosage is 3 to 6 mg of beta carotene (the equivalent of 5,000-10,000 units of vitamin A activity) per day.

Vitamin D is usually in strengths from 50 to 100 international units (IU), it can be found as a soft gel, capsules, tablets and liquids (7). The 2010 recommended daily allowance (RDA) is 600 IU for those 1-71 years of age and 800 IU for those over 71 years of age. For immune function, the following doses have been taken by mouth: 40 IU of vitamin D3 daily for 20 years to 100,000 IU of vitamin D3 bimonthly for 12 months, or 10,000 IU daily. For the treatment of respiratory infections, 2000 IU per kilogram body weight has been taken by mouth daily for three days. For viral infections, 800 IU of vitamin D has been taken by mouth daily for two years, followed by 2000 IU of vitamin D daily for 12 months. For viral infection, 60,000 IU has been taken by mouth weekly for six weeks.

Vitamin B6 refers to a group of chemically similar compounds which can be interconverted in biological systems. Vitamin B6 is part of the vitamin B group of essential nutrients. Its active form, pyridoxal 5-phosphate serves as a coenzyme in some 100 enzyme reactions in amino acid, glucose and lipid metabolism (10). Vitamin B6 is widely distributed in foods in both its free and bound forms. Cooking, storage, and processing losses of vitamin B6 vary and in some foods may be more the 50% depending on the form of vitamin present in the food. The current Estimated Average Requirements (EARs) for vitamin B6 for women and men ages 14 and up increase with age from 1.0 to 1.3 mg/day and from 1.1 to 1.4 mg/day, respectively; the Recommended Dietary Allowance (RDA) increase with age from 1.2 to 1.5 and from 1.3 to 1.7 mg/day, respectively. It has been reported the B vitamins bolster immune response to fight infection and chronic disease (11).

Garlic (Allium sativum) is one of the oldest medicinal plants used by different cultures (3). The oldest reports of health promoting properties of garlic dated back to 16^{th} century BC, when over 20 ailments were purported from Egypt to be efficiently cured by garlic. Garlic stimulates the immune system and acts as a natural antibiotic, not harmful to the friendly bacteria flora. Many laboratory studies have confirmed the antibacterial, antifungal, antivirus, immune-stimulating, and antioxidant properties of garlic. In 1990, the US National Cancer Institute concluded garlic may be a food with cancer-preventive properties.

It has been reported that a composition and a method for stimulating or enhancing the immune system in a human being (2). The composition comprises a mixture of Manapol (a trademark of Carrington Laboratories, Inc., containing acetylated mannans primarily in the form of mannose), beta-1,3-D-glucan, arabinogalactan, elderberry extract, (standardized to about 30% anthocyanins), zinc gluconate and allicin. The composition taken as a food supplement is particularly useful against infectious diseases and acts as an anti-bacterial, anti-viral or anti-fungal agent.

The reports cited above have showed that the ingredients of vitamin C, zinc, vitamin D3, vitamin B6, and garlic, each ingredient alone or in combination with some other materials have been used to stimulate or enhance the immune systems of the human body and to treat the common cold. However, there is no report indicating the use of an alternative composition comprising an effective amount of vitamin C, zinc, vitamin A, vitamin D3, vitamin B6, and garlic in the treatment of bacterial or viral infection.

The present invention provides a pharmaceutical composition comprising an effective amount of vitamin C, zinc, vitamin D3, vitamin A, vitamin B6, and garlic that is able to treat bacterial or viral infection in a patient. In on embodiment, the pharmaceutical composition is an oral composition. In another embodiment the amount of vitamin C is 1100 mg, the amount of zinc is 55 mg, the amount of vitamin A is 50,000 IU, the amount of vitamin D3 is 200 IU, the amount of vitamin B6 35 mg, and garlic is 35IU, in the composition, and the composition in tablet form.

The present invention also provides a method for treating bacterial or viral infection in a patient by orally administering a composition to the patient, wherein the composition comprising an amount of vitamin C, zinc, vitamin A, vitamin D3, Vitamin B6, and garlic effective to reduce the symptoms of bacterial and/or viral infections. In one embodiment, the viral infection is common cold and influenza. In another embodiment, the amount of vitamin C is 1100 mg, D3 is 200 IU, zinc 55 mg, garlic 35 IU, vitamin A 10,000 IU, B6 35 mg, in the composition, and the composition is in the tablet form. In another embodiment, the method further comprises an intervention such as adding a proper diet containing vegetables, and exercising.

As used herein, “a” or “an” means one or more (or at least one)

As used herein, “patient” means either a human being, either adult or children using the oral composition.

### REFERENCES

1. Samenvatting, U.S. Pat. No. 9,616,124

2. Kumar et al., US 20070154581

3. Yakoot, M and Salem, A Efficacy and safety of a multiherbal formula with vitamin C and zinc (Immumax) in the management of the common cold. International Journal of General Medicine 2011:4, 45-51.

4. Mayo Clinic Free E-Newsletter for dosing of vitamin C.

5. Mayo Clinic Free E-Newsletter for dosing of zinc

6. Mayo Clinic Free E-Newsletter for dosing of vitamin A

7. Mayo Clinic Free E-Newsletter for dosing of vitamin D

8. Mayo Clinic Free E-Newsletter for dosing of vitamin B6

9. Nahas, R. and Balla A. Complementary and alternative medicine for prevention and treatment of the common cold. Can. Fam. Physician 2011: 57, 31-36

10. Allan G. M. and Arroll, B Prevention and treatment of the common cold: Making sense of the evidence. CMAJ 2014:186(3), 190-199

11. Micronutrient Information Center, Linus Pauling Institute, Oregon State University, Corvallis, Oreg., May 2014, for “Vitamin B6” (https://lpi.oregonstate.edu/mic/vitamins/vitamins/B6) retrieved on Oct. 2, 2017

12. Phillip, John, Natural News.Com Oct. 13, 2012 “B vitamin bolster immune response to fight infection and chronic disease.

What is claimed is:

1. A pharmaceutical composition comprising an amount of vitamin C, zinc, vitamin A, vitamin D3, vitamin B6, and garlic effective to treat bacterial and/or viral infections.

2. The pharmaceutical composition according to claim 1, the composition is an oral composition.

3. The pharmaceutical composition according to claim 1: wherein the amount of vitamin C is 1100 mg, zinc is 55 mg, vitamin A 50,000 IU, vitamin D3 200 IU, vitamin B6 is 35 mg., and garlic is 35 IU, and wherein the composition is in tablet form.

4. The pharmaceutical composition according to claim 1 wherein the viral infections is colds or influenza.

5. A method for treating bacterial and/or viral infections by orally administering a pharmaceutical composition comprising an amount of vitamin C, zinc, vitamin A, vitamin D3, vitamin B6, and garlic, to a patient effective to treat bacterial and/or viral infections.

6. The method for treating bacterial and/or viral infections according to claim 5, wherein the amount of vitamin C 1100 mg, zinc 55 mg, vitamin A 50,000 IU, vitamin D3 200 IU, vitamin B6 35 mg, and garlic is 35 IU and wherein the composition is in tablet form.

7. The method for treating bacterial and/or viral infections according to claim 5, wherein the viral infection is colds or influenza

8. The method for treating bacterial and/or viral infections according to claim 5, the method further combines with a intervention of using a proper diet containing vegetables and exercising.
